(19) United States
(12) Patent Application Publication
(10) Pub. No.: US 2020/0237695 A1
(43) Pub. Date: Jul. 30, 2020

(54) LEVOTHYROXINE LIQUID FORMULATIONS

(71) Applicant: Hikma Pharmaceuticals USA Inc., Eatontown, NJ (US)

(72) Inventors: Gurmukh Das Chanana, Bedford, OH (US); Ragheb M. AbuRmaileh, Bedford, OH (US)

(21) Appl. No.: 16/776,001

(22) Filed: Jan. 29, 2020

Related U.S. Application Data
(60) Provisional application No. 62/798,639, filed on Jan. 30, 2019.

(51) Int. Cl. A61K 31/197; A61K 47/12; A61K 47/18; A61K 47/10; A61J 1/06

(52) U.S. Cl. CPC A61K 31/197; A61K 47/12; A61J 1/065; A61K 47/10; A61K 47/183

(57) ABSTRACT

A stable liquid formulation for parenteral administration that includes levothyroxine or a pharmaceutically acceptable salt thereof. The formulation includes a low concentration of an antioxidant, an amino acid as a pH adjuster and a stabilization agent in an aqueous solution that has a pH of about 9 to about 11.5. The parenteral liquid formulation is a stable formulation that is supplied as a ready-to-use or ready-to-administer product in a container or vial.

This application claims the benefit of U.S. provisional application Ser. No. 62/798,639 filed Jan. 30, 2019, the contents of which are incorporated herein in their entirety by reference.

### FIELD

The invention relates to a stable liquid formulation containing levothyroxine as an active ingredient and, in particular, a parenteral liquid formulation containing levothyroxine and an antioxidant that is stable.

### BACKGROUND

Levothyroxine, chemically known as 4-(4-hydroxy-3,5-diiodophenoxy)-3,5-diiodo-L-phenylalanine (IUPAC Name. (2S)-2-amino-3-[4-(4-hydroxy-3,5-diiodophenoxy)-3,5-diiodophenyl]propanoic acid), is used as a thyroid hormone replacement therapy in patients with reduced or absent thyroid function, for instance, the condition of myxedema coma. Levothyroxine is typically used in the sodium salt form. However, stability of levothyroxine sodium is affected when it is exposed to high humidity, light or high temperature. Solutions of levothyroxine sodium are also known to be less stable at acidic pH, although degradation impurities can form at basic pH. These properties of the drug have resulted in alternative formulations of levothyroxine sodium to combat the stability disadvantages that are triggered by conditions of high humidity and temperature.

Levothyroxine sodium is conventionally available in an injectable form as a lyophilized product containing 100 mcg, 200 mcg or 500 mcg per unit. The lyophilized injection product is a preservative-free lyophilized powder that includes levothyroxine sodium, mannitol, tribasic sodium phosphate, and sodium hydroxide. Due to the product being lyophilized, it must be reconstituted with sodium chloride solution to prepare an injectable solution suitable for administration, for example, a solution containing levothyroxine sodium at a concentration of 20 mcg/mL, 40 mcg/mL or 100 mcg/mL. The reconstituted solutions are administered shortly after being formed. Lyophilized powders offer the disadvantage of time-consuming reconstitution steps and introduce potential impurities with the use of external components and the potential for error in mixing and measuring such components.

There remains a need for a ready-to-use injectable formulations of levothyroxine that offer long-term storage stability. The present invention provides simplistic stable liquid formulations for parenteral administration of levothyroxine sodium that minimize the number of ingredients and exhibit enhanced storage stability.

### SUMMARY

Described herein are stable parenteral liquid formulations of levothyroxine or salts thereof that include an antioxidant of glutamic acid, monothioglycerol or a combination thereof, and optionally along with an amino acid pH adjuster (e.g., arginine) and propylene glycol. The formulations have a basic pH in the range of about 9 to about 11.5, and preferably about 9.5 to about 11. The formulations exhibit improved stability over long term and accelerated storage conditions, for example, such that no individual degradation product or impurity is formed and present at more than 1 weight percent based on the total weight of the formulation, excluding baseline impurity measurement upon forming the formulation, after storage at 40° C. for 3 months or more. In another example, the formulations are stable for 3 months or more at 25° and 40° C. characterized by about 95% or more, or about 98% or more, of the initial concentration of levothyroxine (e.g., levothyroxine sodium) remaining present in the formulation. In yet another example, the formulations are stable for 3 months or more at 25° and 40° C. characterized by a total impurities content of about 3 weight percent or less based on the total weight of the formulation, excluding baseline total impurity measurement upon forming the formulation.

In a first aspect, there is disclosed a parenteral liquid formulation that includes levothyroxine or a pharmaceutically acceptable salt thereof and an antioxidant selected from the group consisting of about 0.005 to about 0.06 weight percent by volume of glutamic acid, about 0.001 to about 0.02 or about 0.001 to about 0.03 weight percent by volume of monothioglycerol, and a combination thereof. The parenteral liquid formulation has a pH of about 9.0 to about 11.5 and the parenteral liquid formulation is stable for 3 months or more at 40° C., 25° C. or 5° C. Relative humidity at the 40° and 25° C. storage temperatures can be 75% and 60%, respectively, and maintained for the entire storage timer period. Stability of the parenteral liquid formulation can be characterized in that the liquid formulation contains not more than about 1.0% of an individual impurity as formed after forming the formulation, excluding baseline impurity measurement of such an individual impurity upon forming the formulation, over a storage time period of 3 months or more at 40° C., 25° C. or 5° C.

In an example of aspect 1, the levothyroxine or a pharmaceutically acceptable salt thereof is levothyroxine sodium.

In another example of aspect 1, the levothyroxine sodium is present at a concentration of from about 20 mcg/mL to about 125 mcg/mL, or about 100 mcg/mL.

In another example of aspect 1, the levothyroxine sodium is present at a concentration of from about 20 mcg/mL to about 1 mg/mL, about 100 mcg/mL to about 500 mcg/mL, or about 400 mcg/mL to about 800 mcg/mL.

In another example of aspect 1, the levothyroxine sodium is present at a concentration of about 500 mcg/mL and the parenteral formulation is a subcutaneous parenteral formulation for subcutaneous administration.

In another example of aspect 1, the glutamic acid is glutamic acid monosodium monohydrate.

In another example of aspect 1, the glutamic acid is present at about 0.01 to about 0.04 weight percent by volume.

In another example of aspect 1, the formulation contains an antioxidant that consists of a glutamic acid, for example, a glutamic acid is the sole antioxidant in the parenteral liquid formulation and monothioglycerol is present at 0%.

In another example of aspect 1, the monothioglycerol is present at a concentration of from about 0.0025 to about 0.03 weight percent by volume, about 0.0025 to about 0.01 weight percent by volume, about 0.005 weight percent by volume, or about 0.02 or 0.025 weight percent by volume.

In another example of aspect 1, the formulation contains an antioxidant that consists of monothioglycerol, for example, monothioglycerol is the sole antioxidant in the parenteral liquid formulation and glutamic acid is present at 0%.

In another example of aspect 1, the formulation further includes an amino acid as a pH adjuster. The formulation can contain a pH adjuster that consists of an amino acid, for example, the amino acid is the sole pH adjuster in the formulation.

In another example of aspect 1, the amino acid is arginine.

In another example of aspect 1, the arginine is present at about 1 mg/mL to about 15 mg/mL about 2.5 mg/mL to about 12/5 mg/mL, or about 5 mg/mL to about 8 mg/mL.

In another example of aspect 1, the formulation further includes propylene glycol or polyethylene glycol.

In another example of aspect 1, the propylene glycol or polyethylene glycol is present at about 10 to about 60 weight percent by volume, about 25 to about 60 weight percent by volume, about 25 to about 45 weight percent by volume, or about 25 or more, about 30 or more, or about 35 or more weight percent by volume.

In another example of aspect 1, the pH of the formulation is about 9.5 to about 11.0, or about 9.5 to about 10.5.

In another example of aspect 1, the liquid parenteral formulation contains not more than about 1.0% of an individual impurity as formed after forming the formulation after storage for 3 months or more at 40° C., 25° C. or 5° C. Relative humidity at the 40° and 25° C. storage temperatures can be 75% and 60%, respectively, and maintained for the entire storage timer period.

In another example of aspect 1, the parenteral liquid formulation is a ready-to-use or ready-to-administer formulation.

In another example of aspect 1, the formulation is supplied or contained in a glass vial comprising a gas head space, the gas in the head space comprising not more than 5% by volume of oxygen. The glass vial can optionally have a coating on an inner surface that contacts the formulation.

In another example of aspect 1, the gas in the head space includes not more than 1% by volume of oxygen.

In another example of aspect 1, the glass vial is amber or flint color.

In another example of aspect 1, the formulation retains about 95% or more of the initial concentration of levothyroxine or a pharmaceutically acceptable salt thereof after storage for 3 months or more at 40° C. at a relative humidity of 75% or at 5° C.

In another example of aspect 1, the formulation retains about 98% or more, or about 99% or more of the initial concentration of levothyroxine or a pharmaceutically acceptable salt thereof after storage for 3 months or more at 40° C. at a relative humidity of 75% or at 5° C.

In a second aspect, there is disclosed a parenteral liquid formulation that includes about 20 mcg/mL to about 125 mcg/mL of levothyroxine sodium, about 5.0 mg/mL to about 15.0 mg/mL of arginine, about 10 to about 60 weight percent by volume of propylene glycol and about 0.005 to about 0.06 weight percent by volume of glutamic acid. The formulation has a pH of about 9.0 to about 11.5 and the parenteral liquid formulation is stable for at least 3 months or 3 months or more at 40° C. at a relative humidity of 75%, 25° C. at a relative humidity of 60% or at 5° C. Stability of the parenteral liquid formulation can be characterized in that the liquid formulation contains not more than about 1.0% of an individual impurity as formed after forming the formulation, excluding baseline impurity measurement of such an individual impurity upon forming the formulation, over a storage time period of 3 months or more at 40° C., 25° C. or 5° C.

In an example of aspect 2, the glutamic acid is glutamic acid monosodium monohydrate.

In another example of aspect 2, the glutamic acid is present at about 0.01 to about 0.04 weight percent by volume.

In another example of aspect 2, the glutamic acid is the sole antioxidant in the parenteral liquid formulation such that the antioxidant present in the formulation consists of glutamic acid, for example, glutamic acid monosodium monohydrate.

In another example of aspect 2, the liquid parenteral formulation contains not more than about 1.0% of an individual impurity as formed after forming the formulation after storage for 3 months or more at 40° C. at a relative humidity of 75%.

In another example of aspect 2, the liquid parenteral formulation retains about 95% or more of the initial concentration of levothyroxine or a pharmaceutically acceptable salt thereof after storage for 3 months or more at 40° C. at a relative humidity of 75%.

In another example of aspect 2, the parenteral liquid formulation is a ready-to-use or ready-to-administer formulation supplied or contained in a glass vial.

In a third aspect, there is disclosed a parenteral liquid formulation that includes about 20 mcg/mL to about 125 mcg/mL of levothyroxine sodium, about 5.0 mg/mL to about 15.0 mg/mL of arginine, about 10 to about 60 weight percent by volume of propylene glycol, about 0.005 to about 0.04 weight percent by volume of glutamic acid, and about 0.001 to about 0.02 or 0.001 to about 2 weight percent by volume of monothioglycerol. The formulation has a pH value in the range of about 9.0 to about 11.5 and the parenteral liquid formulation is stable for 3 months or more at 40° C. at a relative humidity of 75%.

In an example of aspect 3, the glutamic acid is glutamic acid monosodium monohydrate.

In another example of aspect 3, the glutamic acid is present at about 0.01 to about 0.04 weight percent by volume.

In another example of aspect 3, the monothioglycerol is present at a concentration of from about 0.0025 to about 0.01 weight percent by volume.

In another example of aspect 3, the liquid parenteral formulation contains not more than about 1.0% of an individual impurity as formed after forming the formulation after storage for 3 months or more at 40° C. at a relative humidity of 75%.

In another example of aspect 3, the liquid parenteral formulation retains about 95% or more of the initial concentration of levothyroxine or a pharmaceutically acceptable salt thereof after storage for 3 months or more at 40° C. at a relative humidity of 75%.

In another example of aspect 3, the parenteral liquid formulation is a ready-to-use or ready-to-administer formulation supplied in a glass vial.

In a fourth aspect, there is disclosed a parenteral liquid formulation that includes about 20 mcg/mL to about 1 mg/mL of levothyroxine sodium, about 1.0 mg/mL to about 15.0 mg/mL of arginine, and about 0.001 to about 0.02 weight percent by volume of ascorbic acid. The formulation has a pH value in the range of about 9.0 to about 11.5 and the parenteral liquid formulation is stable for 1 month or more at 40° C. at a relative humidity of 75%, 25° C. at a relative humidity of 60% or at 5° C. Stability of the parenteral liquid formulation is characterized in that the liquid formulation contains not more than about 1.0% of an individual impurity as formed after forming the formulation, excluding baseline impurity measurement of such an individual impurity upon forming the formulation, over a storage time period of 3 months or more at 40° C., 25° C. or 5° C.

In an example of aspect 4, the parenteral liquid formulation includes propylene glycol present at about 10 to about 60 weight percent by volume or about 20 or more weight percent by volume.

In another example of aspect 4, the ascorbic acid is the sole antioxidant in the parenteral liquid formulation or the antioxidant consists of ascorbic acid.

In another example of aspect 4, the increase of total impurity content of the parenteral liquid formulation includes 3 percent or less as compared to the initial total impurity content after formation of the formulation and prior to storage for the at least 1 month at 40° C. at a relative humidity of 75%, 25° C. at a relative humidity of 60% or at 5° C. Total impurity of the parenteral liquid formulation includes all impurities formed after formation of the formulation, excluding baseline total impurity measurement upon forming the formulation.

In another example of aspect 4, the liquid parenteral formulation retains about 98% or more of the initial concentration of levothyroxine or a pharmaceutically acceptable salt thereof after storage for 2 months at 40° C. at a relative humidity of 75%.

In a fifth aspect, there is disclosed a parenteral liquid formulation containing, for example consisting essentially of, about 20 mcg/mL to about 125 mcg/mL of levothyroxine sodium or about 500 mcg/mL of levothyroxine sodium, about 1.0 mg/mL to about 15.0 mg/mL of arginine, about 25 to about 60 weight percent by volume of propylene glycol, about 0.0025 to about 0.02 or about 0.03 weight percent by volume of monothioglycerol, and wherein the parenteral liquid formulation has a pH of about 9.0 to about 11.5 and the parenteral liquid formulation is stable for at least 2 months at 40° C. or 3 months at 40° C.

In an example of aspect 5, the monothioglycerol is present at a concentration of from about 0.005 to about 0.03 weight percent by volume based on the total volume of the formulation, for example, about 1 weight percent by volume, about 2 weight percent by volume or about 2.5 weight percent by volume.

In another example of aspect 5, the liquid parenteral formulation contains not more than about 1.0% of an individual impurity after storage for 3 months at 40° C.

In another example of aspect 5, the liquid parenteral formulation contains not more than about 1.0% or not more than about 0.5% of an individual impurity after storage for 3 months at 5° C.

In another example of aspect 5, the liquid parenteral formulation contains not more than about 0.5% or not more than about 0.25% of an individual impurity after storage for 3 months at 25° C.

In another example of aspect 5, the liquid parenteral formulation retains about 95% or more, about 98% or more, about 98.5% or more, or about 99% or more of the initial concentration of levothyroxine or a pharmaceutically acceptable salt thereof after storage for 3 months at 5° C., 25° C., or 40° C.

In another example of aspect 5, the increase of total impurity content of the parenteral liquid formulation includes 3 percent or less as compared to the initial total impurity content after formation of the formulation and prior to storage for the at least 1 month at 40° C. at a relative humidity of 75%, 25° C. at a relative humidity of 60%, or at 5° C. Total impurity of the parenteral liquid formulation includes all impurities formed after formation of the formulation, excluding baseline total impurity measurement upon forming the formulation.

In another example of aspect 5, the increase of total impurity content of the parenteral liquid formulation includes 3 percent or less as compared to the initial total impurity content after formation of the formulation and prior to storage for 3 months at 40° C. at a relative humidity of 75%. Total impurity of the parenteral liquid formulation includes all impurities formed after formation of the formulation, excluding baseline total impurity measurement upon forming the formulation

In another example of aspect 5, the parenteral liquid formulation is a ready-to-use or ready-to-administer formulation supplied in a glass vial.

In another example of aspect 5, the levothyroxine sodium is present at a concentration of about 500 mcg/mL and the parenteral formulation is a subcutaneous parenteral formulation for subcutaneous administration.

In another example of aspect 5, the parenteral formulation is free of a chelating agent, for example, no chelating agent is present in the parenteral formulation.

Any one of the above aspects (or examples of those aspects) may be provided alone or in combination with any one or more of the examples of that aspect discussed above; e.g., the first aspect may be provided alone or in combination with any one or more of the examples of the first aspect discussed above; and the second aspect may be provided alone or in combination with any one or more of the examples of the second aspect discussed above; and so-forth.

Additional features and advantages will be set forth in the detailed description which follows, and in part will be readily apparent to those skilled in the art from that description or recognized by practicing the embodiments as described herein, including the detailed description which follows, and the claims. It is to be understood that both the foregoing general description and the following detailed description are merely exemplary, and are intended to provide an overview or framework to understanding the nature and character of the claims.

### DETAILED DESCRIPTION

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment.

Herein, when a range such as 5-25 (or 5 to 25) is given, this means preferably at least or more than 5 and, separately and independently, preferably not more or less than 25. In an example, such a range defines independently 5 or more, and separately and independently, 25 or less.

The present disclosure is directed to stable liquid formulations for parenteral administration that include levothyroxine or a pharmaceutically acceptable salt thereof and a low concentration of an antioxidant, for example, glutamic acid, monothioglycerol or a combination thereof. The formulations can optionally include an amino acid as a pH adjuster and propylene glycol in an aqueous solution that has a pH in the range of about 9 to about 11.5.

The formulations of levothyroxine are ideally ready-to-use or ready to administer formulations for parenteral administration without the need for reconstitution. A ready-to-use formulation is a liquid stored in a pharmaceutically suitable container, for example, a glass vial or plastic intravenous bag. A ready-to-use or ready-to-administer formulation is a sterile, liquid injectable formulation not requiring reconstitution before use, for example, a lyophilized powder, such that the formulation can directly administered or further diluted if present as a concentrated solution. For example, a ready-to-administer formulation can be include the required concentration and volume in the final container such as a syringe or injector whereas a ready-to-use preparation can be at the required concentration and a volume in a container that may be transferred to a final administration device such as a syringe or infusion bag for administration to a patient. Parenteral administration may be performed by subcutaneous injection, intramuscular injection, or intravenous injection by means of a syringe, optionally a pen-like syringe. Diluents can include, for instance, fluids suitable for parenteral administration such as water for injection, sodium chloride or dextrose solutions.

The liquid parenteral formulations of the present disclosure are stable or exhibit stability when stored, which includes formulation properties that may be affected by storage conditions, for example, active ingredient strength or concentration, impurities (e.g., individual components and total), visual appearance characteristics (e.g., color, clarity, cloudy, haze, precipitates, etc.) and viscosity. Storage conditions that may affect stability can include, for example, storage temperature, humidity (e.g., relative), light exposure and storage time period.

In one or more embodiments, stability can include the amount of total impurities, inclusive of degradation products, that are formed after formation of the levothyroxine formulation for a specified period of time at specified storage conditions (e.g., temperature, humidity) minus the initial total impurities as measured following formation, for example, within the first 24 hours of formation (i.e. the baseline or initial impurity measurement). In one or more embodiments, the formed levothyroxine formulations of the present invention are stored in a pharmaceutically acceptable container within the first hour, about 2 hours, about 6 hours, about 12 hours or about 24 hours after formation.

In one or more embodiments, a liquid parenteral levothyroxine formulation includes a formulation that retains about 95% or more, about 96% or more, about 97% or more, about 98% or more, about 98.5% or more, or about 99% or more of the initial concentration of levothyroxine or pharmaceutically acceptable salt thereof in the formulation after storage under standard (e.g., 25° C.) or accelerated conditions (e.g., 40° C.). Initial concentration of levothyroxine or a pharmaceutically acceptable salt can be measured shortly after formation and filling of the formulation into a pharmaceutically acceptable container (e.g., a vial) prior to storage. For example, filling of the formulation can be within 24 hours of formation of the formulation. In one or more embodiments, a stable levothyroxine formulation includes a formulation that contains about 0.25% or less, about 0.5% or less, about 0.75% or less, or about 1.0% or less of an individual impurity (e.g., a degradation impurity or levothyroxine-related degradation impurity) formed after formation of the formulation and present after storage under standard or accelerated conditions (e.g., about 25° C. and about 40° C.) for about 1, about 2, about 3, or about 3 or more months. Any measured individual impurity for purposes of measuring stability of a formulation herein does not include any impurity present in any ingredient prior to formation of the levothyroxine formulation. That is, as used herein, an impurity or impurities in an invented formulation refers to any impurity, including a degradation product, formed after formation of the formulations.

In one or more embodiments, the formulations contain liothyronine as an impurity at not more than a measured concentration at a predetermined temperature and time period. For example, the formulation can contain not more than 1.0% liothyronine, not more than about 0.8% liothyronine, not more than about 0.6% liothyronine, not more than about 0.5% liothyronine, not more than about 0.4% liothyronine, not more than about 0.30% liothyronine, not more than about 0.2% liothyronine, or not more than about 0.1% liothyronine, as measured after storage of the formulation at about 25° C. for a period of 3 months. In another example, the formulation can contain not more than about 1.5% liothyronine, not more than about 1.25%, not more than about 1.0%, not more than about 0.8%, not more than about 0.6%, not more than about 0.5%, not more than about 0.45%, or not more than about 0.4% liothyronine, as measured after storage of the formulation at about 40° C. for a period of 3 months.

In one or more embodiments, a stable levothyroxine formulation includes a formulation that contains about 0.5% or less, about 1.0% or less, about 1.5% or less, or about 2.0% or less of total impurities or levothyroxine degradation product present after storage under standard or accelerated conditions. Impurities and degradation products, whether individual or total, can be measured by conventional methods, for example, liquid chromatography (HPLC). In an example, a stable levothyroxine formulation includes about 1.0% or less, about 0.75% or less, about 0.65% or less, about 0.5% or less or about 0.4% or less of total impurities after storage of the formulation at about 25° C. for a period of 3 months. In another example, a stable levothyroxine formulation includes about 4% or less, about 3% or less, about 2.75% or less, about 2.5% or less or about 2% or less of total impurities after storage of the formulation at about 40° C. for a period of 3 months.

In one or more embodiments, a liquid parenteral levothyroxine formulation includes a formulation that is stable for about 3 months or more, about 6 months or more, about 9 months or more, or about 12 months or more when stored at an accelerated temperature of about 25° or 40° C. In one or more embodiments, a liquid parenteral levothyroxine formulation includes a formulation that is stable for about 6 months or more, about 12 months or more, about 18 months or more, or about 24 months or more when stored at about room temperature (i.e. 25° C.).

The formulations of the present disclosure contain, as the active ingredient, levothyroxine or any pharmaceutically acceptable salt thereof. In some embodiments, the formulations preferably contain levothyroxine or any pharmaceutically acceptable salt thereof as the sole active ingredient characterized in that no other active ingredients are present in the formulation. In one example, the formulation contains levothyroxine sodium. In another example, the levothyroxine sodium can be levothyroxine sodium pentahydrate. The levothyroxine or salt thereof, for instance levothyroxine sodium, can be present in the formulation at a concentration of about 10 mcg/mL (micrograms/milliliter) or more, about 20 mcg/mL or more, about 25 mcg/mL or more, or about 50 mcg/mL or more. In one or more embodiments, levothyroxine (e.g., levothyroxine sodium) can be present in the formulation at a concentration of about 1 mg/mL or less, 750 mcg/mL or less, 500 mcg/mL or less, about 400 mcg/mL or less, about 300 mcg/mL or less, about 200 mcg/mL or less, or about 150 mcg/mL or less.

In some embodiments, the liquid parenteral formulations contain a concentration of about 20 mcg/mL, about 40 mcg/mL, about 100 mcg/mL, about 200 mcg/mL, about 500 mcg/mL or about 1 mg/mL of levothyroxine or a pharmaceutically acceptable salt thereof. In one or more embodiments, the liquid parenteral formulations contain about 100 mcg, about 200 mcg or about 500 mcg of levothyroxine or a pharmaceutically acceptable salt thereof per storage container (e.g., vial).

The formulations can be supplied or stored in any suitable volume for parenteral administration. In one or more embodiments, the formulation volume (e.g., amount of liquid in a storage container) is about 0.5 mL or more, about 0.75 mL or more, about 1 mL or more, about 2 mL or more, about 3 mL or more, about 5 mL or more, about 10 mL or more, or about 25 mL or more. For example, the formulation volume can be about 1 mL to about 500 mL, about 1 mL to about 250 mL, about 1 mL to about 100 mL, about 2 mL to about 50 mL, about 2 mL to about 25 mL, or about 3 mL to about 10 mL. In some embodiments, the formulation volume is about 1 mL, about 2 mL, about 3 mL, or about 5 mL. Appropriate-sized containers for storing formulation volumes can be determined by one of ordinary skill in the art.

The formulations for parenteral administration can be stored in or supplied in any suitable container. For example, the formulation can be in a container that includes, but is not limited to, a vial, ampoule, bag (IV bag), bottle, or syringe (e.g., pre-filled syringe or component of an auto-injector). The container can be made of any suitable material, for instance, glass, plastic, or rubber. Prior to filling the formulation in a container, the container is preferably sterile and has been subjected to a sterilization process prior to filing with the sterile formulations of the invention. Containers are sealed as typical in the industry, for example, with the use of a lid, cap, closure, stopper and the like. The containers also can be coated or treated with one or more components to reduce reaction with ingredients of the formulation. For example, a container surface in contact with the formulation can be coated with silicon or a vial with a treated inner surface for storing the formulation can be used. To shield the formulation from exposure to light, a container can optionally be opaque or tinted with a color, and preferably stored in a box for transport or shelving. For instance, amber or flint colored vials are suitable containers.

The formulations further include an antioxidant, either as the sole antioxidant or in combination with another antioxidant, for example, for a total of two or three antioxidants in the formulation. The antioxidant serves to aid in the stabilization of the liquid levothyroxine formulation. In one or more embodiments, the antioxidant is a glutamic acid. Glutamic acid can be present in various forms, for example, any suitable hydrate. An example of glutamic acid can include glutamic acid monosodium monohydrate.

Glutamic acid or variation thereof (e.g., hydrate, salt) can be present in the formulation at suitable concentrations for providing stability to the liquid formulation. For example, the glutamic acid can be present at a concentration of about 0.005 or more weight percent by volume, about 0.01 or more weight percent by volume, about 0.015 or more weight percent by volume, about 0.02 or more weight percent by volume, about 0.025 or more weight percent by volume, 0.03 or more weight percent by volume, 0.035 or more weight percent by volume or about 0.04 or more weight percent by volume, wherein weight percent by volume is based on the total volume of the formulation. In another example, the glutamic acid can be present at a concentration in the range of about 0.005 to about 0.06 weight percent by volume, about 0.01 to about 0.04 weight percent by volume, about 0.015 to about 0.035 weight percent by volume or about 0.015 to about 0.03 weight percent by volume, or about 0.0175, about 0.02, about 0.0225, about 0.025 or about 0.0275 weight percent by volume, wherein volume is based on the total volume of the formulation. In one embodiment, the formulation contains a glutamic acid (e.g., glutamic acid monosodium monohydrate) present at a concentration of about 0.02 weight percent by volume or about 0.04 weight percent by volume. In yet another example, a glutamic acid is the sole antioxidant in the formulation in any of the above noted concentrations such that the formulation consists of a single antioxidant.

In one or more embodiments, the antioxidant in the formulations is monothioglycerol. Monothioglycerol can be present in the formulation at suitable concentrations for providing stability to the liquid formulation. For example, the monothioglycerol can be present at a concentration of about 0.001 or more weight percent by volume, about 0.002 or more weight percent by volume, about 0.003 or more weight percent by volume, about 0.004 or more weight percent by volume, about 0.0045 or more weight percent by volume, 0.005 or more weight percent by volume, about 0.0055 or more weight percent by volume, about 0.006 weight percent by volume, about 0.01 weight percent by volume, about 0.0125 weight percent by volume, about 0.015 weight percent by volume, about 0.0175 weight percent by volume, about 0.02 weight percent by volume, about 0.0225 weight percent by volume, about 0.025 weight percent by volume, about 0.0275 weight percent by volume, or about 0.03 or more weight percent by volume, wherein weight percent by volume is based on the total volume of the formulation. In another example, the monothioglycerol can be present at a concentration in the range of about 0.001 to about 0.03 weight percent by volume, about 0.002 to about 0.025 weight percent by volume, about 0.003 to about 0.02 weight percent by volume or about 0.035 to about 0.015 weight percent by volume, or about 0.004, about 0.005, about 0.006, about 0.007, about 0.008, about 0.009, about 0.01, about 0.011, about 0.012, about 0.013 or about 0.014 weight percent by volume, wherein volume is based on the total volume of the formulation. In another example, the monothioglycerol can be present at a concentration in the range of about 0.001 to about 0.03 weight percent by volume, about 0.002 to about 0.025 weight percent by volume, about 0.003 to about 0.02 weight percent by volume or about 0.035 to about 0.015 weight percent by volume, or about 0.02, about 0.015, about 0.01, about 0.008, about 0.006, about 0.005 or about 0.004 weight percent by volume, wherein volume is based on the total volume of the formulation. In one embodiment, the formulation contains monothioglycerol present at a concentration of about 0.005, about 0.0075, about 0.01 about 0.0125, about 0.015, about 0.02, about 0.025 or about 0.03 weight percent by volume based on the total volume of the formulation. In yet another example, monothioglycerol is the sole antioxidant in the formulation in any of the above noted concentrations such that the formulation consists of a single antioxidant of monothioglycerol. In some embodiments, the parenteral formulations containing monothioglycerol as an antioxidant can be free of a chelating agent (e.g., EDTA) such that no chelating agent is present or detectable in the formulation.

In one or more embodiments, the antioxidant of the formulation is a combination of two antioxidants. The combination can include a glutamic acid and monothioglycerol. Glutamic acid can be present in various forms, for example, any suitable hydrate, for providing stability to the liquid formulation. An example of glutamic acid can include glutamic acid monosodium monohydrate. For example, the glutamic acid can be present at a concentration of about 0.005 or more weight percent by volume, about 0.01 or more weight percent by volume, about 0.015 or more weight percent by volume, about 0.02 or more weight percent by volume, about 0.025 or more weight percent by volume, 0.03 or more weight percent by volume, 0.04 or more weight percent by volume or about 0.06 weight percent by volume, wherein weight percent by volume is based on the total volume of the formulation. In another example, the glutamic acid can be present at a concentration in the range of about 0.005 to about 0.06 weight percent by volume, about 0.01 to about 0.04 weight percent by volume, about 0.015 to about 0.035 weight percent by volume or about 0.015 to about 0.03 weight percent by volume, or about 0.0175, about 0.02, about 0.0225, about 0.025 or about 0.0275 weight percent by volume, wherein volume is based on the total volume of the formulation. Monothioglycerol can be present in the formulation at suitable concentrations for providing stability to the liquid formulation. For example, the monothioglycerol can be present at a concentration of about 0.001 or more weight percent by volume, about 0.002 or more weight percent by volume, about 0.003 or more weight percent by volume, about 0.004 or more weight percent by volume, about 0.0045 or more weight percent by volume, 0.005 or more weight percent by volume, about 0.0055 or more weight percent by volume, about 0.006 weight percent by volume, about 0.01 weight percent by volume, or about 0.02 or more weight percent by volume, wherein weight percent by volume is based on the total volume of the formulation. In another example, the monothioglycerol can be present at a concentration in the range of about 0.001 to about 0.02 weight percent by volume, about 0.002 to about 0.015 weight percent by volume, about 0.003 to about 0.01 weight percent by volume or about 0.035 to about 0.009 weight percent by volume, or about 0.004, about 0.0045, about 0.005, about 0.0055, about 0.006, about 0.007 or about 0.008 weight percent by volume, wherein volume is based on the total volume of the formulation. In one embodiment, the formulation contains a glutamic acid (e.g., glutamic acid monosodium monohydrate) present at a concentration of about 0.02 weight percent by volume and monothioglycerol present at a concentration of about 0.005 weight percent by volume. In another embodiment, the formulation contains a glutamic acid (e.g., glutamic acid monosodium monohydrate) present at a concentration of about 0.04 weight percent by volume and monothioglycerol present at a concentration of about 0.005 weight percent by volume. In yet another example, the combination of a glutamic acid and monothioglycerol represents the sole antioxidants in the formulation in any of the above noted concentrations such that the formulation consists of only these two antioxidants.

The formulations can further include one or more pH adjusters, for example, a single pH adjuster. The pH adjuster can be an amino acid, which can be the sole amino acid and pH adjuster in the formulation. The pH adjuster serves to aid in adjusting the pH of the aqueous formulation and selectively the solubilization of levothyroxine or any salt thereof in the liquid formulation. In one or more embodiments, the pH adjuster is arginine. In water, a concentration of arginine from about 1 mg/mL to about 20 mg/mL adjust the pH of water to about 10.3 to about 11. Arginine exhibited a surprising ability to result in levothyroxine solubilization at concentrations of above 500 mcg/mL in aqueous solutions. Not all amino acid solutions provide solubilization of levothyroxine at a concentration of 500 mcg/mL or more, for example, aspartic acid at concentrations of 1 mg/mL and 4 mg/mL in water exhibited a levothyroxine solubilization of 450 mcg/mL and 100 mcg/mL, respectively.

The pH adjuster (e.g., arginine) can be present in the formulation at a concentration of about 1 mg/mL (milligrams/milliliter) or more, about 2.5 mg/mL or more, or about 5 mg/mL or more. In one or more embodiments, pH adjuster can be present in the formulation at a concentration of about 15 mg/mL or less, about 12.5 mg/mL or less, about 10 mg/mL or less, about 9 mg/mL or less, or about 8 mg/mL or less. In another example, arginine is the sole amino acid pH adjuster in the formulation in any of the above noted concentrations such that the formulation consists of a single amino acid pH adjuster. In one or more embodiments, arginine as a pH adjuster can be present in the parenteral formulation, for instance as the sole amino acid pH adjuster, in an amount in the range of about 2.5 mg/mL to about 15 mg/mL, about 5 mg/mL to about 10 mg/mL, or about 6 mg/mL, about 7 mg/mL, about 7.5 mg/mL, about 8 mg/mL or about 9 mg/mL.

The formulation can have any suitable basic pH. In an example, the formulation can have a pH of about 9 or more, about 9.2 or more, about 9.4 or more, about 9.6 or more, about 9.8 or more, about 10 or more, or about 10.2 or more. In another example, the formulation can have a pH of about 11.5 or less, about 11.3 or less, about 11.1 or less, about 11 or less, about 10.9 or less, about 10.8 or less, about 10.7 or less, about 10.6 or less, or about 10.5 or less.

In one or more embodiments, the formulation can have a pH in the range of about 9 to about 11.5, about 9 to about 11, about 9.2 to about 10.8, about 9.2 to about 10.8, about 9.4 to about 10.8, about 9.6 to about 10.8, about 9.8 to about 10.8, about 10 to about 10.8, about 10 to about 10.7, about 10 to about 10.5, or about 10.2 to about 10.6.

In some embodiments, the liquid parenteral formulations contain a concentration of about 5 mg/mL, about 6 mg/mL, about 6.5 mg/mL, about 7 mg/mL, about 7.5 mg/mL, about 8 mg/mL, or about 8.5 mg/mL of a pH adjuster (e.g., arginine). In one or more embodiments, the liquid parenteral formulations contain about 35 mg, about 37.5 mg or about 40 mg of an amino acid, such as arginine, per storage container (e.g., vial).

The formulations can further include a stabilization agent. The stabilization agent can be a glycol, for example, propylene glycol, polyethylene glycol. The stabilization agent serves to aid in reducing degradation products in the aqueous formulations and selectively retain the concentration of levothyroxine or any salt thereof in the liquid formulation over a period of time. In one or more embodiments, the stabilization agent is propylene glycol.

The stabilization agent (e.g., propylene glycol) can be present in the formulation at a concentration of about 10 weight percent by volume or more, about 20 weight percent by volume or more, about 25 weight percent by volume or more, or about 30 weight percent by volume or more, wherein volume is based on the total volume of the formulation. In one or more embodiments, the stabilization agent can be present in the formulation at a concentration of about 60 weight percent by volume or less, about 50 weight percent by volume or less, about 45 weight percent by volume or less, about 40 weight percent by volume or less, or about 35 weight percent by volume or less, wherein volume is based on the total volume of the formulation. In another example, the propylene glycol is the sole glycol stabilization agent in the formulation in any of the above noted concentrations such that the formulation consists of a single glycol stabilization agent. As used herein, the concentration of the stabilization agent can be expressed in volume percent per total volume of the formulation in place of weight percent per total volume of the formulation.

In some embodiments, the liquid parenteral formulations contain a concentration of about 20 to about 45 weight percent by volume, about 25 to about 40 weight percent by volume, about 30 to about 38 weight percent by volume, or about 32, about 34, about 35 or about 36 weight percent by volume of a stabilization agent (e.g., propylene glycol), wherein volume is based on the total volume of the formulation. In one or more embodiments, the parenteral formulations contain only one stabilization agent in the above concentration or amount ranges. For example, propylene glycol can be present as the sole stabilization agent in the parenteral formulations at an amount of about 25 weight percent by volume, 30 weight percent by volume, 35 weight percent by volume or about 40 weight percent by volume.

Optional ingredients, such as diluents, salts, buffers, tonicity agents, and preservatives, can be provided to the formulation at any stage in its preparation.

In an example parenteral formulation, the formulation can contain levothyroxine or a salt thereof in an amount of 20 mcg/mL, 40 mcg/mL, 100 mcg/mL or 500 mcg/mL, arginine as a pH adjuster in an amount of about 2.5 mg/mL to about 12.5 mg/mL, propylene glycol as a stabilization agent in an amount of about 25 to about 40 weight percent by volume based on the total volume of the formulation, monothioglycerol in an amount of about 0.005, about 0.0075, about 0.01 about 0.0125, about 0.015, about 0.02, or about 0.025 weight percent by volume based on the total volume of the formulation, and water for injection. In another example, the parenteral formulation can contain essentially levothyroxine or a salt thereof in an amount of 20 mcg/mL, 40 mcg/mL, 100 mcg/mL or 500 mcg/mL, arginine as a pH adjuster in an amount of about 6 mg/mL to about 10 mg/mL (e.g., about 8 mg/mL), propylene glycol as a stabilization agent in an amount of about 30 to about 40 (e.g., about 35) weight percent by volume based on the total volume of the formulation, monothioglycerol in an amount of about 0.0025 to about 0.03 (e.g., about 0.005, about 0.01, about 0.025) weight percent by volume based on the total volume of the formulation, and water for injection.

It is another object of this disclosure to prevent loss of stability in a levothyroxine liquid formulation by: a) avoiding cavitation and/or head space formation during preparation of the liquid composition, or b) storing the liquid formulation in a container with a head space that contains oxygen. For example, the head space of a container can contain 5 percent or less of oxygen, with the remaining portion of the gas head space being an inert gas such as argon or nitrogen.

The formulations of the present disclosure are suitable for parenteral administration, for example, to a mammal to treat or prevent a disease or condition. Preferably, the mammal is a human. The noted disease or condition is treatable by the administration of levothyroxine or a pharmaceutically acceptable salt thereof. In an example, the disease or condition is hypothyroidism or myxedema coma.

In order to demonstrate the practice of the present invention, the following examples have been prepared and tested. The examples should not, however, be viewed as limiting the scope of the invention. The claims will serve to define the invention.

### EXAMPLES

### Example 1

This example demonstrates the stability of exemplary formulations including levothyroxine sodium, arginine, propylene glycol, two antioxidants (glutamic acid and monothioglycerol) and water. Separate samples containing the formulation of Table 1 below were prepared at the specified concentrations in an aqueous solution. 5 mL of each sample was filled into vials, type 1, borosilicate, amber glass, and stoppered with under nitrogen.

TABLE 1

| Ingredient | Concentration | |
|---|---|---|
| Levothyroxine sodium | 100 | mcg/mL |
| Arginine | 7.5 | mg/mL |
| Propylene glycol | 30 | volume percent by volume |
| Glutamic acid | 0.02 | weight percent by volume |
| Monothioglycerol | 0.005 | weight percent by volume |
| Head space nitrogen | 5 | percent |

Glutamic acid supplied as glutamic acid monosodium monohydrate. Samples of the formulation were stored at 5°, 25° C. at a relative humidity (RH) of 60% and 40° C. at a relative humidity of 75%. At set periods of storage time, the samples were analyzed by HPLC and measurements of the formulations were taken as shown in Table 2 below. The relative response time (RRT) for impurities is shown. The HPLC conditions were as follows:

Column: Intersil ODS 2 250 mm×4.6 mm, 5 μm, 150 A, P/N 5020; Mobile phase A: 1 mL of trifluoroacetic acid (TFA) in 1000 mL purified water; Mobile Phase B: acetonitrile; Diluent: 0.1 M methanolic sodium hydroxide solution; Column temperature: 25° C.; Flow rate: 0.8 mL/min; Injection volume: 5-50 μL; Autosampler temperature: ambient; Separation mode: gradient; Gradient program:

| Time (minutes) | % Mobile phase A | % Mobile phase B |
|---|---|---|
| 0 | 92 | 8 |
| 25 | 8 | 92 |
| 30 | 8 | 92 |
| 35 | 92 | 8 |
| 40 | 92 | 8 |

The relative response time (RRT) for liothyronine to levothyroxine was approximately 0.93.

TABLE 2

| | | | | | | Total |
|---|---|---|---|---|---|---|
| | | | | | | Impurities |
| | Storage | | | | | including |
| | Temp | | | RRT | RRT | degradation |
| Formulation | (° C.) | pH | Assay | 0.66 | 0.93 | products |
| Initial | N/A | 10.38 | 100 | <LOQ | <LOQ | 0.05 |
| | | | | | | (baseline) |
| 1 month | 25 | | 102.6 | 0.14 | <LOQ | 0.2 |
| | 40 | | 101.5 | 0.36 | 0.19 | 0.69 |
| 2 months | 25 | 10.33 | 101.7 | 0.15 | <LOQ | 0.21 |
| | 40 | 10.36 | 99.3 | 0.59 | 0.33 | 1.26 |
| 3 months | 5 | 10.36 | 101.2 | <LOQ | <LOQ | 0.05 |
| | 25 | 10.26 | 100.6 | 0.20 | <LOQ | 0.25 |
| | 40 | 10.33 | 98.1 | 0.88 | 0.47 | 1.66 |

As can be seen from Table 2, the formulation retains 100% and 98.1% of the initial concentration of levothyroxine sodium after storage for 3 months at 25° and 40° C., respectively. The formulations also demonstrate that no individual impurity or degradation product becomes present at about 1.0% or more after storage for 3 months at 40° C. Total impurities in the formulations remained at 1.26 and 1.66 after storage for 2 and 3 months at 40° C., respectively. The results for individual impurities and total impurities as determined by HPLC show the formulation is stable. For example, the formulation exhibits a total impurity content of 1.75 percent or less and an individual impurity content of 1 percent or less after storage for 3 months at 40° C.

### Example 2

This example demonstrates the stability of exemplary formulations including levothyroxine sodium, arginine, propylene glycol, a single antioxidant (glutamic acid) and water. Separate samples containing the formulation of Table 3 below were prepared at the specified concentrations in an aqueous solution. 5 mL of each sample was filled into vials, type 1, borosilicate, amber glass, and stoppered with under nitrogen.

TABLE 3

| Ingredient | Concentration | |
|---|---|---|
| Levothyroxine sodium | 100 | mcg/mL |
| Arginine | 7.5 | mg/mL |
| Propylene glycol | 30 | volume percent by volume |
| Glutamic acid | 0.02 | weight percent by volume |
| Head space nitrogen | 5 | percent |

Glutamic acid supplied as glutamic acid monosodium monohydrate. Samples of the formulation were stored at 25° C. and 60% RH and 40° C. and 75% RH. At set periods of storage time, the samples were analyzed by HPLC and measurements of the formulations were taken as shown in Table 4 below. The relative response time (RRT) for impurities is shown. The HPLC conditions were the same as Example 1.

TABLE 4

| | | | | | | Total |
|---|---|---|---|---|---|---|
| | | | | | | Impurities |
| | | | | | | including |
| | Storage | | | | | degradation |
| | Temp | | | RRT | RRT | products |
| Formulation | (° C.) | pH | Assay | 0.66 | 0.93 | (wt. %) |
| Initial | N/A | 10.34 | 100 | | <LOQ | <LOQ |
| | | | | | | (baseline) |
| 1 month | 25 | | 103.5 | 0.15 | <LOQ | 0.21 |
| | 40 | | 102.5 | 0.39 | 0.2 | 0.73 |
| 2 months | 25 | 10.3 | 102.3 | 0.19 | | 0.44 |
| | 40 | 10.27 | 99.8 | 0.63 | | 0.86 |
| 3 months | 5 | 10.34 | 102 | <LOQ | | 0.05 |
| | 25 | 10.31 | 102.6 | 0.21 | <LOQ | 0.26 |
| | 40 | 10.31 | 98.4 | 0.92 | 0.49 | 2.08 |

As can be seen from Table 4, the formulation retains 100% and 98.4% of the initial concentration of levothyroxine sodium after storage for 3 months at 25° and 40° C., respectively. The formulations also demonstrate that no individual impurity or degradation product becomes present at about 1.0% or more after storage for 3 months at 40° C. Total impurities in the formulations remained at 0.86 and 2.08 after storage for 2 and 3 months at 40° C., respectively. The results for individual impurities and total impurities as determined by HPLC show the formulation is stable. For example, the formulation exhibits a total impurity content of 2.5 percent or less and an individual impurity content of 1 percent or less after storage for 3 months at 40° C.

### Example 3

Impact of Glycols on Stabilization

This example demonstrates the stability of exemplary formulations including levothyroxine sodium, arginine, propylene glycol and water. Comparison formulations including levothyroxine sodium, arginine, polyethylene glycol and water are shown. Separate samples containing the formulations of Table 5 below were prepared at the specified concentrations in an aqueous solution. 5 mL of each sample was filled into vials, type 1, borosilicate, amber glass, and stoppered with under nitrogen.

TABLE 5

| Ingredient | Formulation 1 | | Formulation 2 | | Formulation 3 | |
|---|---|---|---|---|---|---|
| Levothyroxine | 100 | mcg/mL | 100 | mcg/mL | 100 | mcg/mL |
| sodium | | | | | | |
| Arginine | 7.5 | mg/mL | 7.5 | mg/mL | 7.5 | mg/mL |
| Polyethylene | | | 30% | vol./vol. | | |
| Glycol 300 | | | | | | |
| Propylene | | | | | 30% | vol./vol. |
| Glycol | | | | | | |

| | | | |
|---|---|---|---|
| Water | q.s. to 1 mL | q.s. to 1 mL | q.s. to 1 mL |

Samples of the formulation were stored 25° C. and 60% RH and 40° C. and 75% RH. At set periods of storage time, the samples were analyzed by HPLC and measurements of the formulations were taken as shown in Table 6 below. The HPLC conditions were the same

TABLE 6

| | | | | Percent Total | |
|---|---|---|---|---|---|
| | Percent Assay | | | Degradation | |
| | | 4 months | 4 months | 4 months | 4 months |
| | Initial | (25° C.) | (40° C.) | (25° C.) | (40° C.) |
| Formulation 1 | 103.5 | 96.7 | 77.6 | 11.5 | 33.5 |
| Formulation 2 | 102.2 | 97.4 | 91.7 | 17.2 | 26.5 |
| Formulation 3 | 103.9 | 102.7 | 93.2 | 6.9 | 15.6 |

As can be seen from Table 6, propylene glycol demonstrates increased formulation stability as compared to formulations with no glycol component or polyethylene glycol. For example, propylene glycol exhibited higher concentrations of levothyroxine sodium after storage for 4 months at 25° and 40° C. as compared to the use of polyethylene glycol. In yet another example, propylene glycol exhibited lower concentrations of total degradation products after storage for 4 months at 25° and 40° C. as compared to the use of polyethylene glycol.

### Example 4

This example demonstrates the stability of exemplary formulations including levothyroxine sodium, arginine, propylene glycol, one or two antioxidants (glutamic acid and monothioglycerol) and water. Comparison formulations with other antioxidants, cysteine and sodium sulfoxylate, were also tested. Separate samples containing the formulations labeled below were prepared at the specified concentrations in an aqueous solution. 5 mL of each sample was filled into vials, type 1, borosilicate, amber glass, and stoppered with under nitrogen.

Formulations 1 through 5

| Ingredient | Form. 1 | | Form. 2 | | Form. 3 | | Form. 4 | | Form. 5 | |
|---|---|---|---|---|---|---|---|---|---|---|
| Levothyroxine | 100 | mcg/mL | 100 | mcg/mL | 100 | mcg/mL | 100 | mcg/mL | 100 | mcg/mL |
| Sodium | | | | | | | | | | |
| Arginine | 7.5 | mg/mL | 7.5 | mg/mL | 7.5 | mg/mL | 7.5 | mg/mL | 7.5 | mg/mL |
| Propylene | 30 | vol %/vol. | 30 | vol %/vol. | 30 | vol %/vol. | 30 | vol %/vol. | 30 | vol %/vol. |
| Glycol | | | | | | | | | | |
| Glutamic acid | 0.04 | wt %/vol. | 0.02 | wt %/vol. | 0.02 | wt %/vol. | 0.02 | wt %/vol. | 0.02 | wt %/vol. |
| Monothioglycerol | | | | | 0.005 | wt %/vol. | | | | |
| Cysteine | | | | | | | 0.01 | wt %/vol. | 0.1 | wt %/vol. |

Formulations 6 through 8

| Ingredient | Form. 6 | | Form. 7 | | Form. 8 | |
|---|---|---|---|---|---|---|
| Levo- | 100 | mcg/mL | 100 | mcg/mL | 100 | mcg/mL |
| thyroxine | | | | | | |
| Sodium | | | | | | |
| Arginine | 7.5 | mg/mL | 7.5 | mg/mL | 7.5 | mg/mL |
| Propylene | 30 | vol %/vol. | 30 | vol %/vol. | 30 | vol %/vol. |
| Glycol | | | | | | |
| Glutamic | 0.02 | wt %/vol. | 0.02 | wt %/vol. | 0.02 | wt %/vol. |
| acid | | | | | | |
| Sodium | 0.0375 | wt %/vol. | 0.075 | wt %/vol. | 0.15 | wt %/vol. |
| Sulfoxylate | | | | | | |

Formulations 9 through 13

| Ingredient | Form. 9 | | Form. 10 | | Form. 11 | | Form. 12 | | Form. 13 | |
|---|---|---|---|---|---|---|---|---|---|---|
| Levothyroxine | 100 | mcg/mL | 100 | mcg/mL | 100 | mcg/mL | 100 | mcg/mL | 100 | mcg/mL |
| Sodium | | | | | | | | | | |
| Arginine | 7.5 | mg/mL | 7.5 | mg/mL | 7.5 | mg/mL | 7.5 | mg/mL | 7.5 | mg/mL |
| Propylene | 5 | vol %/vol. | 5 | vol %/vol. | 5 | vol %/vol. | 5 | volt %/vol. | 5 | vol %/vol. |
| Glycol | | | | | | | | | | |
| Glutamic acid | 0.04 | wt %/vol. | 0.02 | wt %/vol. | 0.02 | wt %/vol. | 0.02 | wt %/vol. | 0.02 | wt %/vol. |
| Monothioglycerol | | | | | 0.005 | wt %/vol. | | | | |
| Cysteine | | | | | | | 0.01 | wt %/vol. | 0.1 | wt %/vol. |

Formulations 14 through 16

| Ingredient | Form. 14 | | Form. 15 | | Form. 16 | |
|---|---|---|---|---|---|---|
| Levo- | 100 | mcg/mL | 100 | mcg/mL | 100 | mcg/mL |
| thyroxine | | | | | | |
| Sodium | | | | | | |
| Arginine | 7.5 | mg/mL | 7.5 | mg/mL | 7.5 | mg/mL |
| EDTA | 0.02 | wt %/vol. | 0.02 | wt %/vol. | 0.02 | wt %/vol. |
| Propylene | 5 | vol %/vol. | 5 | vol %/vol. | 5 | vol %/vol. |
| Glycol | | | | | | |
| Glutamic | 0.02 | wt %/vol. | 0.02 | wt %/vol. | 0.02 | wt %/vol. |
| acid | | | | | | |
| Sodium | 0.0375 | wt %/vol. | 0.075 | wt %/vol. | 0.15 | wt %/vol. |
| Sulfoxylate | | | | | | |

Samples of the formulation were stored at 25° C. and 60% RH and 40° C. and 75% RH. At set periods of storage time, the samples were analyzed by HPLC and measurements of the formulations were taken as shown in Tables 7 and 8 below. Table 7 represents data after 1 month of storage. Table 8 represents data after 3 months of storage. The pH of the samples in Tables 7 and 8 were all in the range of about 9.5 to about 10.5. The relative response time (RRT) for impurities is shown. The HPLC conditions were the same as Example 1.

TABLE 7

| | | | | | Total |
|---|---|---|---|---|---|
| | | | | | Impurities |
| | | | | | including |
| | | | | | degradation |
| | Temp | % Assay Change | | | products |
| Form. | (° C.) | from Initial | RRT 0.66 | RRT .093 | (wt. %) |
| 1 | 25 | +2.88 | 0.2 | 0.05 | 0.31 |
| | 40 | +1.67 | 0.63 | 0.23 | 0.99 |
| 2 | 25 | +3.48 | 0.15 | <LOQ | 0.21 |
| | 40 | +2.52 | 0.39 | 0.2 | 0.73 |
| 3 | 25 | +2.62 | 0.14 | <LOQ | 0.2 |
| | 40 | +1.54 | 0.36 | 0.19 | 0.69 |
| 4 | 25 | +2.63 | | 0.05 | 0.11 |
| | 40 | +1.07 | | 0.26 | 0.41 |
| 5 | 25 | +0.47 | | 0.1 | 0.97 |
| | 40 | −14.9 | 0.25 | 0.61 | 6.75 |
| 6 | 25 | +1.67 | 0.15 | 0.05 | 0.36 |
| | 40 | −6.34 | 0.34 | 0.34 | 5.65 |
| 7 | 25 | +1.32 | 0.16 | 0.07 | 1.65 |
| | 40 | −8.3 | 0.37 | 0.3 | 4.7 |
| 8 | 25 | −3.59 | 0.15 | 0.14 | 2.55 |
| | 40 | −6.46 | 0.3 | 0.24 | 3.18 |
| 9 | 25 | +0.77 | 0.6 | 0.05 | 0.74 |
| | 40 | −1.77 | 1.66 | 0.25 | 2.11 |
| 10 | 25 | +0.44 | 0.49 | <LOQ | 0.58 |
| | 40 | −1.0 | 1.1 | 0.21 | 1.44 |
| 11 | 25 | +0.11 | 0.4 | | 0.49 |
| | 40 | −0.99 | 0.66 | 0.18 | 1.34 |
| 12 | 25 | +0.55 | | 0.05 | 0.14 |
| | 40 | −1.44 | | 0.26 | 0.43 |
| 13 | 25 | −0.89 | | 0.08 | 1.37 |
| | 40 | −5.68 | | 0.62 | 2.57 |
| 14 | 25 | +0.22 | 0.44 | | 0.53 |
| | 40 | −5.56 | 0.96 | 0.21 | 2.77 |
| 15 | 25 | 0.0 | 0.44 | | 1.42 |
| | 40 | −10.25 | 0.68 | 0.37 | 3.6 |
| 16 | 25 | −1.56 | 0.37 | | 2.0 |
| | 40 | −11.96 | 0.76 | 0.34 | 4.2 |

TABLE 8

| | | | | | Total |
|---|---|---|---|---|---|
| | | | | | Impurities |
| | | | | | including |
| | | | | | degradation |
| | Temp | % Assay Change | | | products |
| Form. | (° C.) | from Initial | RRT 0.66 | RRT .093 | (wt. %) |
| 1 | 5 | +0.95 | <LOQ | <LOQ | 0.06 |
| | 25 | +1.31 | 0.33 | <LOQ | 0.38 |
| | 40 | −1.67 | 0.95 | 0.6 | 2.2 |
| 2 | 5 | +2.04 | <LOQ | <LOQ | 0.05 |
| | 25 | +2.64 | 0.21 | <LOQ | 0.26 |
| | 40 | −1.56 | 0.92 | 0.49 | 2.08 |
| 3 | 5 | +1.19 | <LOQ | <LOQ | 0.05 |
| | 25 | +0.59 | 0.2 | <LOQ | 0.25 |
| | 40 | −1.90 | 0.88 | 0.47 | 1.66 |
| 4 | 5 | +1.79 | 0.05 | <LOQ | 0.10 |
| | 25 | +1.67 | 0.3 | 0.07 | 0.52 |
| | 40 | −4.78 | 1.19 | 0.69 | 2.86 |
| 5 | 5 | +1.07 | <LOQ | <LOQ | 0.77 |
| | 25 | −1.55 | 0.12 | 0.13 | 3.33 |
| | 40 | −21.12 | 0.69 | 1.37 | 23.4 |
| 9 | 5 | +1.44 | 0.16 | <LOQ | 0.22 |
| | 25 | 0.0 | 0.95 | <LOQ | 1.23 |
| | 40 | −7.0 | 3.38 | 0.58 | 4.67 |
| 10 | 5 | +1.44 | 0.14 | <LOQ | 0.20 |
| | 25 | +0.22 | 0.87 | <LOQ | 1.21 |
| | 40 | −4.34 | 2.57 | 0.5 | 3.88 |
| 11 | 5 | +1.32 | 0.13 | <LOQ | 0.19 |
| | 25 | −0.1 | 0.57 | 0.06 | 0.63 |
| | 40 | −4.86 | 2.2 | 0.49 | 3.13 |
| 12 | 5 | +1.33 | 0.16 | <LOQ | 0.29 |
| | 25 | +0.66 | 0.68 | 0.07 | 0.86 |
| | 40 | −6.44 | 2.61 | 0.64 | 4.21 |
| 13 | 5 | +1.33 | 0.08 | <LOQ | 0.50 |
| | 25 | −1.67 | 0.46 | 0.1 | 1.46 |
| | 40 | −27.53 | 1.56 | 1.22 | 22.71 |

The data from Tables 7 and 8 show that formulations 1, 2 and 3 result in reduced levels of individual and total impurities, inclusive of degradation products formed after initial formation of the formulation, as compared to the remaining tested formulations. After 3 months of storage at 5, 25° and 40° C., formulation 1 exhibited a total impurity content of 0.06, 0.38 and 2.2 percent, respectively, and 1 percent or less of any individual impurity. Formulation 2, after 3 months of storage at 5°, 25° and 40° C., exhibited a total impurity content of 0.05, 0.26 and 2.08 percent, respectively, and 1 percent or less of any individual impurity. Similarly, formulation 3 under the same storage conditions exhibited a total impurity content of 0.05, 0.25 and 1.66 percent and 1 percent or less of any individual impurity.

In contrast, formulations 4-5 and 9-16 exhibited one or more individual impurities at 1 percent or more after 3 months of storage at 25° and 40° C. Formulations 4, 5, 12 and 13 show that the use of cysteine as an antioxidant in combination with glutamic acid was less effective at reducing individual and total impurities including degradation products as compared to using glutamic acid alone as the sole antioxidant whether the propylene glycol was at 30 or 5 volume percent by volume. For example, formulations 4, 5, 12 and 13 exhibited a total impurity content of 2.86 percent or more after 3 months of storage at 40° C.

Formulations 1, 2 and 3, after 1 month of storage at 40° C. exhibit a total impurity content of 1 percent or less. Formulations 6-8 and 14-16 show that the use of sodium sulfoxylate as an antioxidant in combination with glutamic acid was less effective at reducing total impurities as compared to using glutamic acid alone as the sole antioxidant whether the propylene glycol was at 30 or 5 volume percent by volume. For example, formulations 6-8 and 14-16 all exhibited a total impurity content of 3 percent or more after 1 month of storage at 40° C. Formulations 14 through 16 also included ethylenediaminetetraacetic acid (EDTA). The addition of EDTA as a chelating did not increase the stability of the formulations.

The data from Tables 7 and 8 show that formulations 1, 2 and 3 result in an increased amount of levothyroxine sodium present after being stored as compared to the remaining tested formulations. After 3 months of storage at 5° and 25° C., formulations 1, 2 and 3 exhibited no reduction in levothyroxine sodium. After 3 months of storage at 40° C., formulations 1, 2 and 3 exhibited a reduction of levothyroxine of 2 percent or less, namely 1.67, 1.56 and 1.9 percent, respectively.

In contrast, formulations 4-5 and 9-16 exhibited a reduction in levothyroxine sodium of 4 percent or more after 3 months of storage at 40° C. or greater than double the reduction of formulations 1, 2 and 3. Formulations 6-8 and 14-16 show that the use of sodium sulfoxylate as an antioxidant in combination with glutamic acid was less effective at reducing levothyroxine sodium loss as compared to using glutamic acid alone as the sole antioxidant whether the propylene glycol was at 30 or 5 volume percent by total volume. For example, after 1 month of storage at 40° C., formulations 6-8 and 14-16 exhibited a levothyroxine sodium loss in the range of 6.34 to 11.96 percent as compared to formulations 1, 2 and 3 that exhibited 0 percent loss of levothyroxine. The inclusion of EDTA in formulations 14 through 16 also appears to provide no benefit to reducing loss of levothyroxine sodium.

The tables further show that the use of cysteine as an antioxidant in combination with glutamic acid was less effective at reducing levothyroxine sodium as compared to using glutamic acid alone as the sole antioxidant whether the propylene glycol was at 30 or 5 volume percent by total volume. For example, formulations 4, 5, 12 and 13 exhibited a reduction in levothyroxine sodium of 4.78 percent or more, and up to 27.53 percent, after 3 month of storage at 40° C.

### Example 5

This example demonstrates the stability of exemplary formulations including levothyroxine sodium, 100 mcg/mL, arginine, propylene glycol, one or two antioxidants (glutamic acid monosodium monohydrate and monothioglycerol) and water. Separate samples containing the formulations labeled below were prepared at the specified concentrations in an aqueous solution. 5 mL of each sample was filled into vials, type 1, borosilicate, amber glass, and stoppered with under nitrogen.

Formulations 1 through 22

| | | Propylene | | |
|---|---|---|---|---|
| | Arginine | Glycol | Monothioglycerol | Glutamic acid |
| Form. | (mg/mL) | (vol %/vol.) | (wt %/vol.) | (wt %/vol.) |
| 1 | 7.5 | 40 | 0.005 | 0.02 |
| 2 | 1 | 10 | 0.005 | 0.02 |
| 3 | 1 | 20 | | 0.02 |
| 4 | 1 | 40 | | 0.02 |
| 5 | 7.5 | 10 | 0.005 | |
| 6 | 7.5 | 40 | 0.005 | |
| 7 | 7.5 | 40 | 0.005 | |
| 8 | 7.5 | 20 | 0.005 | 0.02 |
| 9 | 10 | 20 | 0.005 | |
| 10 | 1 | 40 | 0.005 | |
| 11 | 7.5 | 20 | 0.005 | 0.02 |
| 12 | 10 | 20 | 0.005 | |
| 13 | 10 | 10 | 0.005 | 0.02 |
| 14 | 10 | 10 | | 0.02 |
| 15 | 1 | 20 | 0.005 | |
| 16 | 10 | 40 | 0.005 | 0.02 |
| 17 | 1 | 40 | 0.005 | 0.02 |
| 18 | 10 | 40 | | 0.02 |
| 19 | 7.5 | 20 | 0.005 | 0.02 |
| 20 | 7.5 | 20 | | 0.02 |
| 21 | 7.5 | 20 | | 0.02 |
| 22 | 1 | 10 | 0.005 | |

Samples of the formulation were stored at 25° C. and 60% RH and 40° C. and 75% RH. At set periods of storage time, the samples were analyzed by HPLC and measurements of the formulations were taken as shown in Tables 9 and 10 below. Table 9 represents data after 2 months of storage. Table 10 represents data after 3 months of storage. The pH of the samples in Tables 9 and 10 were all in the range of about 9.5 to about 10.5. The relative response time (RRT) for impurities is shown. The HPLC conditions were the same as Example 1.

TABLE 9

| | | | | | Total |
|---|---|---|---|---|---|
| | | | | | Impurities |
| | | | | | including |
| | | % Assay | | | degradation |
| | Temp | Decrease from | | | products |
| Form. | (° C.) | Initial | RRT 0.66 | RRT 0.93 | (wt. %) |
| 1 | 25 | 0 | <LOQ | 0.057 | 0.272 |
| | 40 | 0 | 0.079 | 0.371 | 1.258 |
| 2 | 25 | 0 | 0.469 | 0.05 | 0.651 |
| | 40 | 0 | 1.344 | 0.29 | 2.231 |
| 3 | 25 | 0 | 0.336 | 0.068 | 0.485 |
| | 40 | 0 | 0.993 | 0.318 | 1.768 |
| 4 | 25 | 0 | 0.253 | 0.184 | 0.721 |
| | 40 | 0 | 0.57 | 0.477 | 1.70 |
| 5 | 25 | 0 | <LOQ | <LOQ | 0.081 |
| | 40 | 0 | 0.24 | 0.286 | 0.963 |
| 6 | 25 | 0 | <LOQ | 0.047 | 0.312 |
| | 40 | 0 | 0.071 | 0.285 | 1.131 |
| 7 | 25 | 0 | <LOQ | 0.048 | 0.317 |
| | 40 | 0 | 0.079 | 0.294 | 1.141 |
| 8 | 25 | 0 | 0.168 | 0.049 | 0.347 |
| | 40 | 0 | 1.13 | 0.357 | 1.992 |
| 9 | 25 | 0 | <LOQ | <LOQ | 0.081 |
| | 40 | 0 | 0.458 | 0.312 | 1.327 |
| 10 | 25 | 0 | 0.132 | 0.055 | 0.387 |
| | 40 | 0 | 0.492 | 0.35 | 1.495 |
| 11 | 25 | 0 | 0.19 | 0.05 | 0.364 |
| | 40 | 0 | 1.465 | 0.35 | 2.339 |
| 12 | 25 | 0 | <LOQ | <LOQ | 0.127 |
| | 40 | 0 | 0.416 | 0.314 | 1.273 |
| 13 | 25 | 0 | 0.064 | 0.048 | 0.243 |
| | 40 | 0 | 0.287 | 0.372 | 1.109 |
| 14 | 25 | 0 | 0.068 | 0.059 | 0.358 |
| | 40 | 0 | 0.298 | 0.379 | 1.144 |
| 15 | 25 | 0 | <LOQ | 0.329 | 0.409 |
| | 40 | 0 | 4.065 | 0.258 | 5.316 |
| 16 | 25 | 0 | <LOQ | 0.056 | 0.273 |
| | 40 | 0 | 0.061 | 0.386 | 1.232 |
| 17 | 25 | 0 | 0.153 | 0.072 | 0.467 |
| | 40 | 0 | 0.588 | 0.416 | 1.795 |
| 18 | 25 | 0 | <LOQ | 0.064 | 0.366 |
| | 40 | 0 | 0.071 | 0.388 | 1.269 |
| 19 | 25 | 0 | 0.184 | 0.049 | 0.363 |
| | 40 | 0 | 0.114 | 0.354 | 1.013 |
| 20 | 25 | 0 | 0.303 | 0.061 | 0.555 |
| | 40 | 0 | 0.133 | 0.357 | 1.043 |
| 21 | 25 | 0 | 0.259 | 0.058 | 0.449 |
| | 40 | 0 | 1.217 | 0.359 | 2.273 |
| 22 | 25 | 0 | <LOQ | 0.46 | 0.542 |
| | 40 | −1.11% | 1.647 | 0.201 | 2.422 |

Total impurities are measured as change from initial impurities upon formation of the formulation.

TABLE 10

| | | | | | Total |
|---|---|---|---|---|---|
| | | | | | Impurities |
| | | | | | including |
| | | % Assay | | | degradation |
| | Temp | Decrease from | | | products |
| Form. | (° C.) | Initial | RRT 0.66 | RRT 0.93 | (wt. %) |
| 1 | 5 | 0 | 0.059 | <LOQ | 0.059 |
| | 25 | 0 | 0.232 | 0.077 | 0.591 |
| | 40 | 0 | 0.772 | 0.547 | 2.637 |
| 2 | 5 | 0 | 0.198 | <LOQ | 0.332 |
| | 25 | 0 | 0.713 | 0.066 | 0.932 |
| | 40 | −3.22% | 2.034 | 0.418 | 3.335 |
| 3 | 5 | 0 | 0.170 | <LOQ | 0.253 |
| | 25 | 0 | 0.533 | 0.090 | 0.820 |
| | 40 | 0 | 1.518 | 0.451 | 2.807 |
| 4 | 5 | 0 | 0.084 | <LOQ | 0.215 |
| | 25 | 0 | 0.445 | 0.244 | 0.960 |
| | 40 | 0 | 0.855 | 0.725 | 2.691 |
| 5 | 5 | 0 | 0.178 | <LOQ | 0.305 |
| | 25 | 0 | 0.634 | 0.051 | 0.873 |
| | 40 | −0.2% | 1.848 | 0.429 | 3.099 |
| 6 | 5 | 0 | 0.052 | <LOQ | 0.179 |
| | 25 | 0 | 0.204 | 0.064 | 0.602 |
| | 40 | 0 | 0.634 | 0.439 | 2.403 |
| 7 | 5 | 0 | 0.063 | <LOQ | 0.201 |
| | 25 | 0 | 0.241 | 0.067 | 0.682 |
| | 40 | 0 | 0.723 | 0.442 | 2.567 |
| 8 | 5 | 0 | 0.140 | <LOQ | 0.222 |
| | 25 | 0 | 0.495 | 0.069 | 0.744 |
| | 40 | 0 | 1.571 | 0.523 | 2.978 |
| 9 | 5 | 0 | <LOQ | <LOQ | 0.137 |
| | 25 | 0 | <LOQ | 0.057 | 0.295 |
| | 40 | 0 | 1.577 | 0.481 | 2.925 |
| 10 | 5 | 0 | 0.049 | <LOQ | 0.177 |
| | 25 | 0 | 0.249 | 0.111 | 0.656 |
| | 40 | 0 | 0.774 | 0.507 | 2.337 |
| 11 | 5 | 0 | 0.157 | <LOQ | 0.286 |
| | 25 | 0 | 0.465 | 0.074 | 0.769 |
| | 40 | 0 | 1.994 | 0.518 | 3.469 |
| 12 | 5 | 0 | <LOQ | <LOQ | 0.127 |
| | 25 | 0 | 0.548 | 0.062 | 0.800 |
| | 40 | 0 | 1.488 | 0.478 | 2.844 |
| 13 | 5 | 0 | <LOQ | <LOQ | 0.081 |
| | 25 | 0 | <LOQ | 0.066 | 0.311 |
| | 40 | 0 | 2.238 | 0.550 | 3.654 |
| 14 | 5 | 0 | <LOQ | <LOQ | 0.185 |
| | 25 | 0 | <LOQ | 0.082 | 0.366 |
| | 40 | −3.05% | 2.165 | 0.576 | 3.740 |
| 15 | 5 | 0 | 0.131 | <LOQ | 0.212 |
| | 25 | 0 | 0.495 | 0.06 | 0.698 |
| | 40 | 0 | 1.820 | 0.345 | 2.967 |
| 16 | 5 | 0 | <LOQ | <LOQ | 0.135 |
| | 25 | 0 | 0.326 | 0.077 | 0.736 |
| | 40 | 0 | 0.779 | 0.566 | 2.604 |
| 17 | 5 | 0 | 0.049 | <LOQ | 0.131 |
| | 25 | 0 | 0.263 | 0.116 | 0.575 |
| | 40 | 0 | 0.859 | 0.792 | 2.700 |
| 18 | 5 | 0 | <LOQ | <LOQ | 0.186 |
| | 25 | 0 | 0.380 | 0.083 | 0.848 |
| | 40 | 0 | 0.916 | 0.561 | 2.744 |
| 19 | 5 | 0 | 0.137 | <LOQ | 0.218 |
| | 25 | 0 | 0.467 | 0.069 | 0.673 |
| | 40 | 0 | 1.524 | 0.529 | 2.950 |
| 20 | 5 | 0 | 0.191 | <LOQ | 0.320 |
| | 25 | 0 | 0.565 | 0.078 | 0.902 |
| | 40 | 0 | 1.726 | 0.533 | 3.299 |
| 21 | 5 | 0 | 0.162 | <LOQ | 0.247 |
| | 25 | 0 | 0.577 | 0.080 | 0.909 |
| | 40 | 0 | 1.754 | 0.536 | 3.453 |
| 22 | 5 | 0 | 0.216 | <LOQ | 0.349 |
| | 25 | 0 | 2.630 | <LOQ | 3.106 |
| | 40 | −3.62% | 2.431 | 0.291 | 3.528 |

### Example 6

This example demonstrates the stability of exemplary formulations including levothyroxine sodium (500 mcg/mL), arginine (7.5 mg/mL), ascorbic acid as an antioxidant (0.2 wt %/vol.) and water. Separate samples containing the formulations labeled below were prepared at the specified concentrations in an aqueous solution. 5 mL of each sample was filled into vials, type 1, borosilicate, amber glass, and stoppered with under nitrogen.

Samples of the formulation were stored at 25° C. and 60% RH and 40° C. and 75% RH. At set periods of storage time, the samples were analyzed by HPLC and measurements of the formulations were taken as shown in Table 11. Table 11 represents data after 1 and 2 months of storage. The pH of the samples in Table 11 were all in the range of about 9.5 to about 10.5. The relative response time (RRT) for impurities is shown. The HPLC conditions were the same as Example 1.

TABLE 11

| | | | | Total |
|---|---|---|---|---|
| | | | | Impurities |
| | | | | including |
| Storage | | % Assay | | degradation |
| Time | Temperature | Decrease from | | products |
| (months) | (° C.) | Initial | RRT (0.93) | (wt. %) |
| 1 | 25 | 0 | 0.111 | 1.1 |
| 1 | 40 | −1.13% | 0.747 | 2.5 |
| 2 | 25 | 0 | 0.129 | — |
| 2 | 40 | −1.04% | 1.316 | — |

The data from Table 11 shows that the use of ascorbic acid at about 0.2 weight percent by volume, wherein volume is based on the total volume of the formulation, as the sole antioxidant in the levothyroxine formulation results in reduced levels of individual and total impurities, inclusive of degradation products formed after initial formation of the formulation. After 1 month of storage at 25° and 40° C., the formulation exhibited a total impurity content of 1.1 and 2.5 percent, respectively, and 1 percent or less of any individual impurity, for example, liothyronine (RRT 0.93). Similarly, after 2 months of storage at 25° C., the formulation exhibited 1% or less of any individual impurity such as liothyronine.

### Example 7

This example demonstrates the stability of exemplary formulations including levothyroxine sodium (500 mcg/mL), arginine (7.5 mg/mL), EDTA (2 mg/mL), 30 (wt %/vol.) propylene glycol, monothioglycerol as an antioxidant (0.02 wt %/vol.) and water. Separate samples containing the formulations labeled below were prepared at the specified concentrations in an aqueous solution. 5 mL of each sample was filled into vials, type 1, borosilicate, amber glass, and stoppered with under nitrogen.

Samples of the formulation were stored at 25° C. and 60% RH. At set periods of storage time, the samples were analyzed by HPLC and measurements of the formulations were taken as shown in Table 12. Table 12 represents data after 1, 2 and 3 months of storage. The pH of the samples in Table 12 were all in the range of about 9.5 to about 10.5. The relative response time (RRT) for impurities is shown. The HPLC conditions were the same as Example 1.

TABLE 12

| | | | | Total |
|---|---|---|---|---|
| | | | | Impurities |
| | | | | including |
| Storage | | % Assay | | degradation |
| Time | Temperature | Decrease from | | products |
| (months) | (° C.) | Initial | RRT (0.93) | (wt. %) |
| 1 | 25 | 0 | 0.024 | 0.7 |
| 2 | 25 | 0 | 0.082 | 5.1 |

### Example 8

This example demonstrates the characteristics of other antioxidants in formulations including levothyroxine sodium (500 mcg/mL), arginine (7.5 mg/mL), EDTA (0.2 wt %/vol.), propylene glycol (30 vol %/vol.) and water. To the separate samples of the formulation, three formulations were prepared each with a different antioxidant. In formulation 1, 0.02 wt %/vol. of butylated hydroxy anisole (BHA) was added. In formulation 2, 0.02 wt %/vol. of butylated hydroxy toluene (BHT) was added. In formulation 3, 0.02 wt %/vol. of propyl gallate was added. 5 mL of each sample was filled into vials, type 1, borosilicate, amber glass, and stoppered with under nitrogen. Formulation 1 containing the BHA antioxidant turned purple upon formation. Formulation 2 containing the BHT antioxidant contained visual particles as the BHT did not fully dissolve. Formulation 3 containing the propyl gallate antioxidant turned yellow within 30 minutes after being formed. None of the three tested antioxidants exhibited initial solution characteristics that warranted further testing as seen with the formulations of the present invention.

### Example 9

This example demonstrates the stability of exemplary formulations including levothyroxine sodium (100 mcg/mL), arginine (8 mg/mL), 35 (vol %/vol.) propylene glycol, monothioglycerol as an antioxidant (0.025, 0.05 and 0.1 wt %/vol.) and water. Separate samples containing the formulations labeled below were prepared at the specified concentrations in an aqueous solution. 5 mL of each sample was filled into vials, type 1, borosilicate, amber glass, and stoppered with under nitrogen.

Formulations 1 through 3

| | | | Propylene | |
|---|---|---|---|---|
| | Levothyroxine | | glycol | Monothio- |
| | Sodium | Arginine | (vol. %/ | glycerol |
| Formulation | (mcg/mL) | (mg/mL) | total vol.) | (wt. %/vol.) |
| 1 | 100 | 8 | 35 | 0.025 |
| 2 | 100 | 8 | 35 | 0.05 |
| 3 | 100 | 8 | 35 | 0.1 |

Samples of the formulation were stored at 25° C. and 60% RH and 40° C. and 75% RH. At set periods of storage time, the samples were analyzed by HPLC and measurements of the formulations were taken as shown in Table 13. Table 13 represents data after 2 months of storage. The pH of the samples in Table 13 were all in the range of about 9.5 to about 10.5. The relative response time (RRT) for impurities is shown. The HPLC conditions were the same as Example 1.

TABLE 13

| | | | | | Total |
|---|---|---|---|---|---|
| | | | | | Impurities |
| | | | | | including |
| | | % Assay | | | degradation |
| | Temp | Decrease from | | | products |
| Form. | (° C.) | Initial | RRT 0.65 | Liothyronine | (wt. %) |
| 1 | 25 | 0 | <LOQ | 0.07 | 0.42 |
| | 40 | −0.9 | 0.26 | 0.39 | 2.02 |
| 2 | 25 | 0 | <LOQ | 0.08 | 1.36 |
| | 40 | −1.57 | 0.22 | 0.44 | 2.99 |
| 3 | 25 | −1.28 | <LOQ | 0.11 | 3.67 |
| | 40 | −4.38 | 0.23 | 0.86 | 6.16 |

The data from Table 13 shows that the use of monothioglycerol at about 0.025 weight percent by volume, wherein volume is based on the total volume of the formulation, as the sole antioxidant in the levothyroxine formulation results in reduced levels of individual and total impurities, inclusive of degradation products formed after initial formation of the formulation. After 2 months of storage at 25° and 40° C., formulation 1 exhibited a total impurity content of 0.42 and 2.02 percent, respectively, and 0.5 percent or less of any individual impurity, for example, liothyronine. Thus, formulation 1 evidences a stable formulation with a respective total impurity content of 0.5 or less and 2.5 percent or less after 2 months of storage at 25° and 40° C.

Table 13 also evidences that monothioglycerol concentrations at 0.05 or more weight percent per total volume increase liothyronine impurity and total impurities including degradation products. For instance, at a monothioglycerol concentration of 0.05 weight percent per volume, the total impurities including degradation products increased to about 3 weight percent per total volume of the formulation after 2 months of storage at 40° C. After 2 months of storage at 40° C. for formulation 3 having a monothioglycerol concentration of 0.1 weight percent per volume, the total impurities including degradation products increased to above 6 weight percent per total volume of the formulation.

It is also shown that at monothioglycerol concentrations at 0.05 or more weight percent per total volume can cause levothyroxine concentrations to decrease. For example, at 0.05 and 0.1 weight percent per total volume of monothioglycerol (i.e. formulations 2 and 3), the concentration of levothyroxine in the formulations decreased 1.57 percent and 4.38 percent, respectively, after 2 months of storage at 40° C.

Many variations and modifications may be made to the above-described embodiments of the disclosure without departing substantially from the spirit and various principles of the disclosure. All such modifications and variations are intended to be included herein within the scope of this disclosure and protected by the following claims.

What is claimed is:

1. A parenteral liquid formulation comprising:
a. levothyroxine or a pharmaceutically acceptable salt thereof;
b. an antioxidant selected from the group consisting of about 0.005 to about 0.06 weight percent by volume of glutamic acid, about 0.001 to about 0.03 weight percent by volume of monothioglycerol, and a combination thereof,
wherein the parenteral liquid formulation has a pH of about 9.0 to about 11.5 and the parenteral liquid formulation is stable for at least 3 months at 40° C.

2. The parenteral liquid formulation of claim 1, wherein levothyroxine or a pharmaceutically acceptable salt thereof is levothyroxine sodium.

3. The parenteral liquid formulation of claim 2, wherein the levothyroxine sodium is present at a concentration of from about 20 mcg/mL to about 125 mcg/mL.

4. The parenteral liquid formulation of claim 2, wherein the levothyroxine sodium is present at a concentration of from about 400 mcg/mL to about 800 mcg/mL.

5. The parenteral liquid formulation of claim 1, wherein the monothioglycerol is present at a concentration of from about 0.005 to about 0.03 weight percent by volume.

6. The parenteral liquid formulation of claim 1, wherein the monothioglycerol is the sole antioxidant in the parenteral liquid formulation.

7. The parenteral liquid formulation of claim 1, further comprising an amino acid pH adjuster.

8. The parenteral liquid formulation of claim 7, wherein the amino acid is arginine.

9. The parenteral liquid formulation of claim 8, wherein the arginine is present at about 1 mg/mL to about 15 mg/mL.

10. The parenteral liquid formulation of claim 1, further comprising propylene glycol at about 10 to about 60 weight percent by volume.

11. The parenteral liquid formulation of claim 1, wherein the pH is about 9.0 to about 11.0.

12. The parenteral liquid formulation of claim 1, wherein the liquid parenteral formulation comprises not more than about 1.0% of an individual impurity after storage for 3 months at 40° C.

13. The parenteral liquid formulation of claim 1, wherein the liquid parenteral formulation comprises not more than about 1.0% of an individual impurity after storage for 3 months at 5° C.

14. The parenteral liquid formulation of claim 1, wherein the parenteral liquid formulation is a ready-to-use or ready-to-administer formulation.

15. The parenteral liquid formulation of claim 14, wherein the formulation is supplied in a glass vial comprising a gas head space, the gas in the head space comprising not more than 5% by volume of oxygen.

16. The parenteral liquid formulation of claim 15, wherein the glass vial is amber or flint color.

17. The parenteral liquid formulation of claim 1, wherein the formulation retains about 95% or more of the initial concentration of levothyroxine or a pharmaceutically acceptable salt thereof after storage for 3 months at 40° C.

18. The parenteral liquid formulation of claim 1, wherein the formulation retains about 98% or more of the initial concentration of levothyroxine or a pharmaceutically acceptable salt thereof after storage for 3 months at 40° C.

19. The parenteral liquid formulation of claim 1, wherein the formulation retains about 98% or more of the initial concentration of levothyroxine or a pharmaceutically acceptable salt thereof after storage for 3 months at 5° C.

20. A parenteral liquid formulation comprising:
a. about 20 mcg/mL to about 125 mcg/mL of levothyroxine sodium;
b. about 1.0 mg/mL to about 15.0 mg/mL of arginine;
c. about 25 to about 60 weight percent by volume of propylene glycol;
d. about 0.0025 to about 0.03 weight percent by volume of monothioglycerol; and
wherein the parenteral liquid formulation has a pH of about 9.0 to about 11.5 and the parenteral liquid formulation is stable for at least 3 months at 40° C.

21. The parenteral liquid formulation of claim 20, wherein the monothioglycerol is present at a concentration of from about 0.005 to about 0.02 weight percent by volume.

22. The parenteral liquid formulation of claim 20, wherein the liquid parenteral formulation comprises not more than about 1.0% of an individual impurity after storage for 3 months at 40° C.

23. The parenteral liquid formulation of claim 20, wherein the liquid parenteral formulation comprises not more than about 1.0% of an individual impurity after storage for 3 months at 5° C.

24. The parenteral liquid formulation of claim 20, wherein the liquid parenteral formulation retains about 95% or more of the initial concentration of levothyroxine or a pharmaceutically acceptable salt thereof after storage for 3 months at 40° C.

25. The parenteral liquid formulation of claim 20, wherein the liquid parenteral formulation retains about 95% or more of the initial concentration of levothyroxine or a pharmaceutically acceptable salt thereof after storage for 3 months at 5° C.

26. The parenteral liquid formulation of claim 20, wherein the parenteral liquid formulation is a ready-to-use or ready-to-administer formulation supplied in a glass vial.
